(19) United States
(12) Patent Application Publication
(10) Pub. No.: US 2023/0381494 A1
(43) Pub. Date: Nov. 30, 2023

(54) ELECTRIC PULSE MASSAGE APPARATUS AND SYSTEM

(71) Applicant: Shenzhen Xiaobin Technology Co., Ltd., Shenzhen (CN)

(72) Inventor: Chaoyun YUE, Shenzhen (CN)

(73) Assignee: Shenzhen Xiaobin Technology Co., Ltd., Shenzhen (CN)

(21) Appl. No.: 17/874,764

(22) Filed: Jul. 27, 2022

(30) Foreign Application Priority Data
May 27, 2022 (CN) 202221326094.8

(51) Int. Cl. A61N 1/04; A61N 1/36

(52) U.S. Cl. CPC A61N 1/0456; A61N 1/0472; A61N 1/36031

(57) ABSTRACT

The utility model discloses an electric pulse massage apparatus and system, the apparatus comprising: A control assembly comprises a first interface unit, a second interface unit, a power chip and a pulse generation unit, wherein the first interface unit is used for accessing an external power supply, the first interface unit is connected to a pulse generation unit through the power chip, the power chip is used for providing a power supply to the pulse generation unit, the pulse unit is connected to a second interface unit. Therefore, the pulse generation unit is arranged in the control assembly to generate an electric pulse to provide the electric pulse to the massage assembly, without depending on the special power supply apparatus, only an external power source needs to be accessed, the use is more convenient, the portability is improved, and the use experience of the user is improved.

### CROSS-REFERENCE TO RELATED APPLICATIONS

The application claims priority to Chinese patent application No. 202221326094.8, filed on May 27, 2022, the entire contents of which are incorporated herein by reference.

### TECHNICAL FIELD

The utility model relates to the field of massage technologies, and in particular, to an electric pulse massage apparatus and system.

### BACKGROUND

In the related technologies, the electric pulse massage is usually a pair of electrode sheets connected to a power supply apparatuss, such as a physiotherapy instrument, and the power supply apparatuss outputs electrical pulses to the human body through a pair of electrode sheets, thereby performing pulse electrical stimulation massage on the human body.

Current power supply apparatus is usually a large-volume and heavy power supply, when a user uses, the electrode sheet needs to be connected to a power supply apparatuss, the power supply apparatuss is adjusted to output an electric pulse, the electric pulse is applied to the human body through the electrode sheet for electric stimulation massage, the power supply apparatuss needs to be adjusted and set on the power supply apparatuss at a time, and the power supply apparatuss is inconvenient to carry.

### SUMMARY

The utility model provides an electric pulse massage apparatus and system, and aims to solve the problem that an existing electric pulse massage apparatus is inconvenient to use.

In the first aspect, the utility model provides an electric pulse massage apparatus, comprising: A control assembly comprises a first interface unit, a second interface unit, a power chip and a pulse generation unit, wherein the first interface unit is used for accessing an external power supply, the first interface unit is connected to a pulse generation unit through the power chip, the power chip is used for providing a power supply to the pulse generation unit, the pulse unit is connected to a second interface unit, and the pulse generation unit is used for generating an electric pulse; The massage assembly is connected to the second interface unit, and the massage assembly is used for receiving the electric pulse and apply the electric pulse to the human body for massage.

Further, the massage assembly comprises a positive electrode sheet, a negative electrode sheet and a connection wire, the positive electrode sheet and the negative electrode sheet are connected to the second interface unit through the connection wire, the positive electrode sheet and the negative electrode sheet are used for being attached to the surface of a human body, and the electric pulse is output to the human body for massage.

Further, the positive electrode plate and the negative electrode plate both comprise a centrifugal film layer, a conductive gel layer, a conductive carbon film layer and a fabric layer, and the centrifugal film layer, the conductive gel layer, the conductive carbon film layer and the fabric layer are sequentially stacked; Wherein, an outer contour of the conductive gel layer matches an outer contour of the fabric layer, an outer contour of the conductive carbon film layer is smaller than an outer contour of the fabric layer, so that a gap is provided between an outer contour of the conductive carbon film layer and an outer contour of the fabric layer, and a width of the gap is 0.8 mm to 1.2 mm.

Further, the massage assembly also comprises a conductor and a conductive coating film, a through hole is formed in the fabric layer and the conductive carbon film layer, the conductor is disposed on the through hole, and the conductive coating film covers the conductor and is connected to the conductive carbon film layer.

Further, the massage assembly comprises a positive electrode ear clip and a negative electrode ear clip and a connection wire, the positive electrode ear clip and the negative electrode ear clip are connected to the second interface unit through the connection wire, the positive electrode ear clip and the negative electrode ear clip are used for being clamped to the human ear, and the electrical pulse is output to the human ear for massage.

Further, the massage assembly further comprises a conductor, the positive electrode ear clip and the negative electrode ear clip both comprise a first clamping piece and a second clamping piece, and the first clamping piece and the second clamping piece can be close to or far away from each other to clamp or loosen a human ear; The first clamping piece and the second clamping piece are both provided with the conductors.

Further, the pulse unit comprises a main control chip and a pulse boost control circuit, the main control chip is connected to the power chip, and the main control chip is connected to the second interface unit by means of the pulse boost control circuit.

Further, the control assembly further comprises a key unit, the key unit is connected to the main control chip, and the key unit is used for triggering a control signal to cause the main control chip to adjust the electric pulse according to the control signal.

In the second aspect, the utility model further provides an electric pulse massage system, comprising: A wearable apparatuss and an electric pulse massage apparatuss, wherein the electric pulse massage apparatuss is the electric pulse massage apparatuss according to the first aspect, the wearable apparatuss is connected to the first interface unit, and the wearable apparatuss is used for providing a power supply.

Further, the wearable apparatus comprises a monitoring module, the monitoring module is in communication connection with the pulse generation unit, the monitoring module is used for monitoring a physiological parameter electric signal of a human body, and the pulse generation unit is used for adjusting an electric pulse according to the physiological parameter electric signal.

Compared with the prior technologies, the utility model has the following beneficial effects: By arranging the control assembly and the massage assembly, the control assembly comprises a first interface unit, a second interface unit, a power chip and a pulse generation unit, the first interface unit is connected to the external power supply, the second interface unit is connected to the massage assembly, power is supplied to the pulse generation unit by using the electric energy provided by the external power supply, the pulse generation unit generates electric pulses to be transmitted to the massage assembly through the second interface unit, and the massage assembly acts on the human body for electric stimulation massage. Therefore, compared with the prior technologies, the electric pulse is generated by the power supply apparatuss which is inconvenient to carry. In the present application, the pulse generation unit is built into the control assembly, and an interface is provided in the control assembly to connect the external power supply and the massage assembly, so that the user does not need to connect to a large bulky special power supply apparatuss when in use, without adjusting an electric pulse on the power supply apparatuss, and can generate an electric pulse by means of the pulse generation unit of the control assembly, and only needs to connect the interface to any external power supply, without carrying bulky special power supply apparatuss, thereby improving portability and improving user experience.

### BRIEF DESCRIPTION OF DRAWINGS

In order to more clearly illustrate the technical solutions of the embodiments of the utility model, the drawings required in the description of the embodiments will be briefly introduced below. Obviously, the drawings in the following description are some embodiments of the utility model. For those of ordinary skill in the technology, other drawings may be obtained according to these drawings without creative efforts.

FIG. 1 shows a schematic diagram of an electric pulse massage system according to the embodiment of the utility model;

FIG. 2 shows a schematic diagram of an electric pulse massage apparatus according to the embodiment of the utility model;

FIG. 3 shows a schematic diagram of a control assembly of an electric massage apparatus according to an embodiment of the utility model;

FIG. 4 shows another schematic diagram of the control assembly of the electric pulse massage apparatus according to the embodiment of the utility model;

FIG. 5 shows a schematic diagram of an electrode sheet of an electric pulse massage apparatus according to an embodiment of the utility model;

FIG. 6 shows an enlarged view of Part A of FIG. 5;

FIG. 7 shows a side view schematic diagram of the electrode sheet of the electric pulse massage apparatus according to the embodiment of the utility model;

FIG. 8 produces an enlarged view of Part B of FIG. 5;

FIG. 9 shows a schematic diagram of a massage assembly of an electric pulse massage apparatus according to an embodiment of the utility model;

FIG. 10 shows a schematic diagram of another massage assembly of the electric pulse massage apparatus according to the embodiment of the utility model;

FIG. 11 shows a schematic diagram of a. wearable apparatus of an electric pulse massage apparatus according to an embodiment of the utility model;

FIG. 12 shows a module schematic diagram of the electric pulse massage apparatus according to the embodiment of the utility model;

10, a Control assembly; 11, a First interface unit; 12, a Second interface unit; 13, a Power chip; 14, a Pulse generation unit; 15, a Key unit; 16, a Main control chip; 17, a Pulse boost control circuit; 20, a Massage assembly; 21, an Electrode sheet; 211, a Conductive gel layer; 212, a Conductive carbon film layer; 213, a Fabric layer; 214, a Conductor; 215, a Conductive coating film; 22, an Electrode ear clip; 23, a Connection wire; 30, a Wearable apparatus.

### DETAILED DESCRIPTION OF THE EMBODIMENTS

The technical solutions in the embodiments of the utility model will be clearly and completely described below with reference to the accompanying drawings in the embodiments of the utility model. Obviously, the described embodiments are some embodiments of the utility model rather than all of the embodiments. All other embodiments obtained by person of ordinary skill in the technology based on the embodiments of the utility model without creative efforts shall fall within the protection scope of the utility model. It should be understood that the terms “comprising” and “including” when used in this specification and the appended brief description of the claims indicate the presence of the described features, integers, steps, operations, elements, and/or assemblies, but do not preclude the presence or addition of one or more other features, integers, steps, operations, elements, assemblies, and/or sets thereof.

It should also be understood that the terminology used in this specification is for the purpose of describing particular embodiments only and is not intended to be limiting of the utility model. As used in this specification and the appended brief description of the claims, the singular forms “a” “an” and “the” are intended to include the plural forms as well, unless the context clearly indicates otherwise.

It should also be further understood that the term “and/or” as used in the specification and the appended brief description of the claims, means any combination of one or more of the associated listed items, and possibly combinations thereof, and includes these combinations.

The embodiments of the utility model provide an electric pulse massage apparatus, which solves the problem that an existing electric pulse massage apparatus needs a dedicated heavy power supply apparatus to provide an electric pulse to cause inconvenience to use. By arranging the pulse unit on the control assembly, it is only necessary to connect the interlace to any external power source without carrying a bulky dedicated power supply apparatus, thereby improving portability.

The technical solution in the embodiments of the utility model is to solve the problem of inconvenience to use, and the general idea is as follows:

The prior technology massage apparatuss requires special power supply equipment to provide low-frequency pulses, such as a physiotherapy instrument, and this type of massage apparatuss is large in size and inconvenient to carry. Therefore, the present application provides a control assembly the control assembly is provided with an interface and a pulse generation unit, the interface is connected to any external power source, for example, a power bank, a power adapter, etc., power is supplied to the massage assembly by using an external power supply, and the pulse generation unit in the control assembly generates an electric pulse to the massage assembly. Therefore, when in use, only any external power source needs to be accessed, the power supply apparatuss with the pulse generation module is not required, the use is convenient, the portability is improved, and the user experience is good.

In order to better understand the above technical solutions, the above technical solutions will be described in detail below with reference to the specification drawings and specific embodiments.

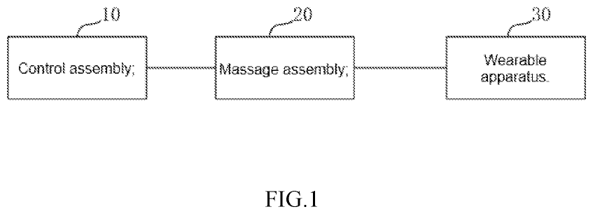

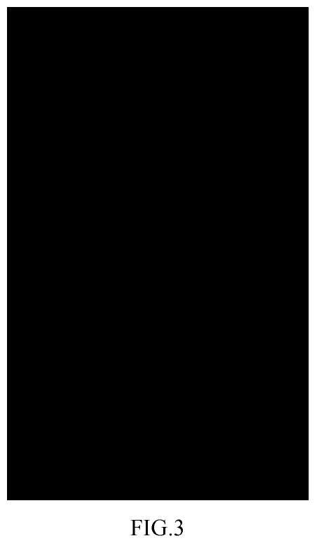

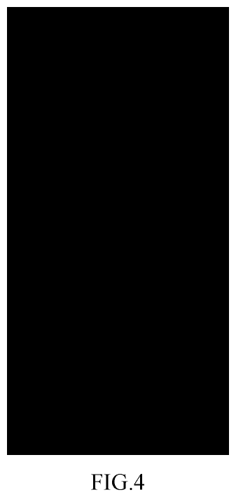

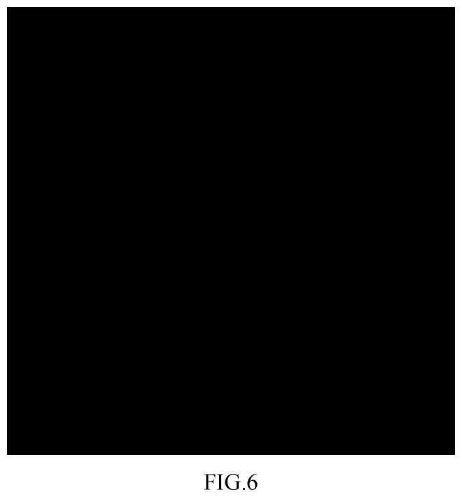

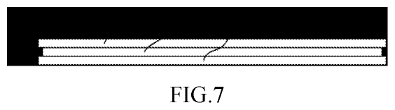

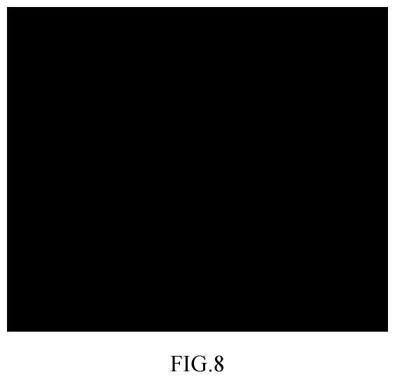

Referring to FIG. 1 to FIG. 11, this embodiment provides an electric pulse massage apparatus, comprising: The control assembly 10 comprises a first interface unit 11, a second interface unit 12, a power chip 13 and a pulse generation unit 14, wherein the first interface unit 11 is used for accessing an external power supply, the first interface unit 11 is connected to the pulse generation unit 14 through the power chip 13, the power chip 13 is used far providing a power supply to the pulse generation unit 14, the pulse unit is connected to the second interface unit 12, and the pulse generation unit 14 is used for generating an electric pulse; The massage assembly 20 is connected to the second interface unit 12, and the massage assembly 20 is used for receiving the electric pulse and apply an electric pulse to the human body for massage.

By implementing the embodiment, the pulse generation unit 14 is arranged in the control assembly 10 to generate an electrical pulse to provide the electric pulse to the massage assembly 20, without depending on the special power supply apparatuss, the use is more convenient, the portability is improved, and the use experience of the user is improved.

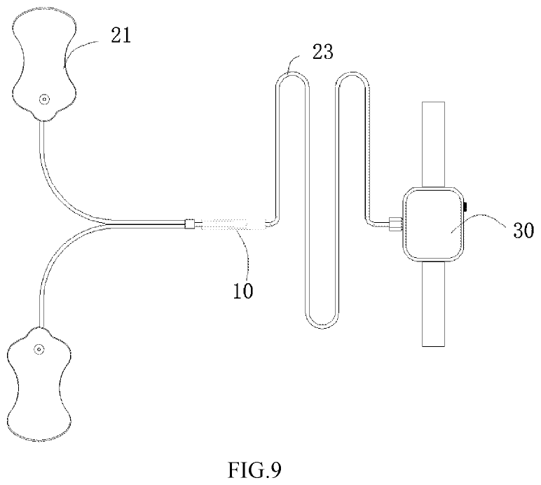

Referring to FIG. 9, in an embodiment, the massage assembly 20 comprises a positive electrode sheet 21 and a negative electrode sheet 21 and a connection wire 23, the positive electrode sheet 21 and the negative electrode sheet 21 are connected to the second interface unit 12 through the connection wire 23, and the positive electrode sheet 21 and the negative electrode sheet 21 are used for being attached to the surface of a human body, and the electric pulse is output to the human body for massage. Specifically, the massage assembly 20 of the present embodiment is composed of two electrode sheets 21 and a connection wire 23, the two electrode sheets 21 are respectively configured as a positive electrode and a negative electrode, and when the electric pulse is transmitted, the current flows from the positive electrode sheet 21 to the human body and returns from the negative electrode sheet 21 to the wearable apparatus 30 to form a conductive loop, and the electric pulse performs electrical stimulation massage in the human body. One side of the electrode sheet 21 is sticky and can be attached to the skin surface of the human body, for example, when the shoulder of the human body is ache, the side of the electrode sheet 21 can be adhered to the shoulder portion for electrical stimulation massage. For another example, when the neck of the human body is ache, the neck of the human body may be adhered to perform electrical stimulation massage on the neck. For another example, when the waist of the human body is ache, the waist of the human body may be adhered to perform electrical stimulation massage on the waist. Of course, in this embodiment, it can be understood that the electrode sheet 21 may also be adhered to other positions of the human body for electrical stimulation massage, which will not be enumerated herein one by one. Therefore, the massage assembly 20 of the present embodiment is adhered to the to-be-massaged part of the human body in a manner of double electrode sheets 21, can adapt to various massage positions of the human body, can massage a plurality of massage parts, is convenient and flexible to use, and has good user experience.

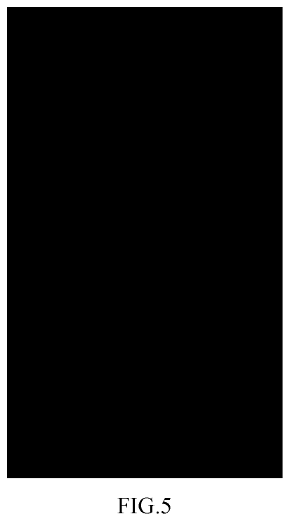

Referring to FIG. 5 to FIG. 8, in this embodiment, the positive electrode sheet 21 and the negative electrode sheet 21 both comprise a centrifugal film layer, a conductive gel layer 211, a conductive carbon film layer 212 and a fabric layer 213, and the centrifugal film layer, the conductive gel layer 211, the conductive carbon film layer 212 and the fabric layer 213 are sequentially stacked; An outer contour of the conductive gel layer 211 matches an outer contour of the fabric layer 213, and an outer contour of the conductive carbon film layer 212 is smaller than an outer contour of the fabric layer 213, so that a gap is provided between an outer contour of the conductive carbon film layer 212 and an outer contour of the fabric layer 213, and a width of the gap is 0.8 mm to 1.2 mm. Specifically, the fabric layer 213 is a substrate of the electrode sheet 21, and is mainly characterized in that other layers are supported, which have certain flexibility, and the material of the fabric layer 213 may be fabric leather, foam or PU\PET\non-woven fabric\cortical layer, etc. The conductive carbon film layer 212 is mainly used for transmitting electrical pulses. The conductive gel layer 211 has a certain viscosity, can be attached to the skin surface of the human body, ensures the reliability of the connection with the human body, and transmits the electric pulse to the human body by using the conductive gel layer 211. The centrifugal film layer is disposed on the surface of the conductive gel layer 211, and mainly functions to protect the conductive gel layer 211, avoid external dust impurities on the conductive gel layer 211, can tear off the centrifugal film layer when in use, and attach the centrifugal film layer to the conductive gel layer 211 after use. In the embodiment, the outer contour of the conductive gel layer 211 matches the outer contour of the fabric layer 213, that is, the surface area of the conductive gel layer 211 is equal to the surface area of the fabric layer 213, and the conductive gel layer 211 can completely cover the fabric layer 213 and the conductive carbon film layer 212.

In the embodiment, the purpose of providing a gap between the outer contour of the conductive carbon film layer 212 and the outer contour of the fabric layer 213 is to avoid the edge wear of the electrode sheet 21, because the electrode sheet 21 is usually adhered to the skin of the human body, or is torn off from the skin of the human body, usually with the fingernail to pick up a warped angle and then tearing, the fingernail will touch the edge of the electrode sheet 21 every time it is used. Therefore, it causes wear on the edge of the electrode sheet 21, the conductive carbon film layer 212 can be separated from the conductive gel layer 211 after long-term use, so that the edge of the conductive carbon film layer 212 is exposed and the coverage of the conductive gel layer 211 is lost, so that the user can generate an edge puncture feeling when in use, and the use experience is poor. Therefore, in order to avoid this situation, a gap is provided between the outer contour of the conductive carbon film layer 212 and the outer contour of the fabric layer 213, and the outer contour of the conductive carbon film layer 212 is relatively small, so that even if the edge flanging of the conductive carbon film layer 212 is not exposed, the conductive carbon film layer 212 can still be covered by the conductive gel layer 211, thereby avoiding the generation of the puncture pain, and improving the user experience. In the embodiment, the width of the gap is correspondingly defined, exemplarily, the width of the gap is 0.8 mm. Exemplarily, the width of the gap is 0.9 mm. Exemplarily, the width of the gap is 0.9 mm. Exemplarily, the width of the gap is 0.9 mm. Exemplarily, the width of the gap is 0.9 mm.

In an embodiment, the massage assembly 20 further comprises a conductor 214 and a conductive coating film 215, a through hole is formed in the fabric layer 213 and the conductive carbon film layer 212, the conductor 214 is disposed on the through hole, and the conductive coating film 215 covers the conductor 214 and is connected to the conductive carbon film layer 212. Specifically, the conductor 214 is used for being connected with the connection wire 23, and serves as a conductive function, so as to transmit the electric pulse to the conductive carbon film layer 212. The conductive coating film 215 is used far covering the conductor 214, because the conductor 214 is disposed in the through hole, the conductor 214 is exposed on the conductive carbon film layer 212, so that the appearance integrity of the conductive carbon film layer 212 is poor. Therefore, in this embodiment, the conductive coating film 215 is provided to cover the conductive coating film 215 an the conductor 214, so that the conductive coating film 215 is integrated with the conductive carbon film layer 212, and on the other hand, the appearance integrity is provided, and on the other hand, the contact area between the conductor 214 and the conductive carbon film layer 212 can be increased by using the conductive coating film 215, thereby improving the conductivity.

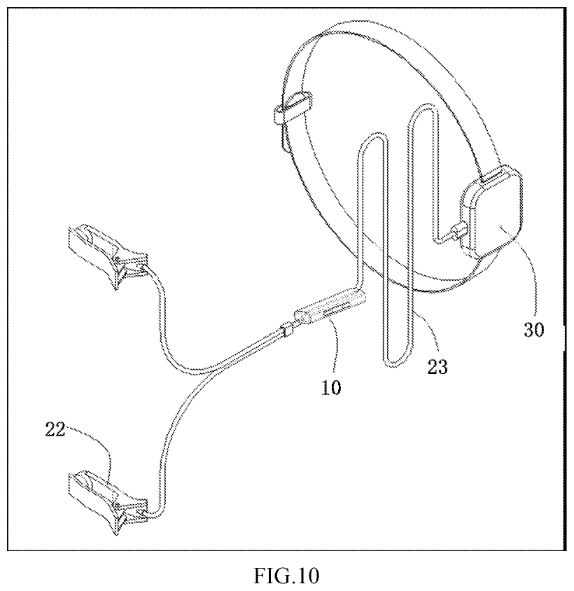

Referring to FIG. 10, in an embodiment, the massage assembly 20 comprises a positive electrode ear clip 22 and a negative electrode ear clip 22 and a connection wire 23, the positive electrode ear clip 22 and the negative electrode ear clip 22 are connected to the second interface unit 12 through the connection wire 23, and the positive electrode ear clip 22 and the negative electrode ear clip 22 are used for being clamped to the ear, and the electric pulse is output to the human ear for massage. Specifically, the electrode ear clip 22 is in the shape of a clip and has two clamping pieces, and the two clamping pieces can be far away from or close to each other, so that the ear of the human body is clamped, and at least position of the electrode ear clip 22 facing the ear is electrically conductive and used for transmitting the electric pulse to the ear. Similarly, the electrode ear clip 22 of the embodiment also a pair, which is a positive electrode ear clip 22 and a negative electrode ear clip 22, respectively, current flows from the positive electrode ear clip 22 to the human body and returns from the negative electrode ear clip 22 to the wearable apparatuss 30 to form a conductive loop, and the electrical pulse performs electrical stimulation massage in the human body. Therefore, the massage assembly 20 of the embodiment is clamped to the ear of the human body through the two electrode ears clips 22, and transmits the electric pulse signal through the ear of the human body to help the human body quickly enter the sleep state.

In an embodiment, the massage assembly 20 further comprises a conductor 214, and both the positive electrode ear clip 22 and the negative electrode ear clip 22 comprise a first clamping piece and a second clamping piece, and the first clamping piece and the second clamping piece can be close to or far away from each other to clamp or loosen a human ear portion; The conductors 214 are disposed on two opposite sides of the first clamping pieces and the second clamping pieces. Specifically, the conductor 214 is provided with two, one conductor 214 is disposed on the first clamping piece, and the other conductor 214 is disposed on the second clamping piece, and the two conductors 214 are disposed opposite to each other. When the electrode ear clip 22 clamps the human ear, the electric pulse is transmitted to the human body through the two conductors 214 The two conductors 214 are used to transmit electrical pulses to achieve bipolar conduction, and the electrical stimulation degree is more appropriate relative to the monopole conduction (ie, a conductor 214), because the contact area between the electrode ear clip 22 and the ear of the monopolar conductive electrode is small, and the generated electrical stimulation is high, which is easy to generate the discomfort of the pricking pain. In this embodiment, the contact area between the electrode ear clip 22 and the ear position can be increased by using the bipolar conductive, thereby reducing the degree of electrical stimulation, avoiding the discomfort of generating the puncture pain, and improving the use experience of the user. Specifically, due to the fact that only one electrode and current flow to the human body through the electrode, the stimulation degree is large, while the bipolar conductive has two electrodes, the current flows to the human body through the two electrodes, the current is shunted by the double electrodes, the current is reduced, and the electrical stimulation degree is reduced.

In this embodiment, the electrical pulse may be adjusted to implement transcranial micro-current stimulation therapy or transcutaneous nerve electrical stimulation therapy. Specifically, cranial electrotherapy stimulation (CES) is a treatment method that is completely different from traditional drug therapy and electrical twitch therapy, and is a neurotransmitter and a hormone that stimulate the brain of the brain and change the brain abnormality of the patient by stimulating the brain with a low-intensity micro-current, so as to cause the brain to excrete a series of neurotransmitters and hormones that are closely linked to diseases such as anxiety, depression, insomnia and the like, so as to achieve the treatment of these diseases. It should be noted that the brain wave abnormality and the central nervous system secretion disorder are the root cause of insomnia, and the CES therapy has an improved effect on regulating abnormal brain activity. The sleep instrument releases micro-ampere-like biological current through the electrode clamped at the earlobe position. The long-term conditioning can gradually recover abnormal brain waves and neurotransmitter secretion to be normal, thereby achieving the effect of assisting in improving sleep. The CES can adopt regular symmetric wave output, so that the treatment accuracy can be greatly improved, and the experience comfort level is higher.

TENS, Transcutaneous Electrical Nerve Stimulation (TENS), is a weak electrical pulse, “close” the pain signal of the brain, and naturally let your body “release enkephalin”, and the enkephalin is a chemical substance with good physical sensation. The non-drug non-invasive pain-relieving method, TENS is stimulated by mild micro current (usually no more than 25 micro-coulombs in unit time) to occlude the transmission of the hyperalgesia nerve signal, thereby achieving the effect of analgesia. The TENS technology refers to a low-frequency pulse massage technology, which is to stimulate a specific low-frequency pulse wave stimulation sensory nerve fiber to achieve the therapy of analgesia through the skill, thereby helping to alleviate muscle pain and fatigue. The TENS low frequency pulse massage technology is adopted to help to alleviate muscle pain and fatigue. The normal teacher-level massage method is clear and meridians, and soothing nerves to quickly recover body vitality.

In this embodiment, the positive electrode ear clip 22 and the negative electrode ear clip 22 are both provided with electromagnetic assembly, the electromagnetic assembly is configured to drive the positive electrode ear clip 22 and the negative electrode ear clip 22 to clamp under magnetic force when the electrical pulse is output, and the positive electrode ear clip 22 and the negative electrode ear clip 22 are released when the electric pulse stops outputting. Specifically, the electrode ear clip 22 is provided with an electromagnetic assembly, the electromagnetic assembly includes a magnetic attraction assembly and a metal assembly, the magnetic attraction assembly is disposed on a clamping plate, and the metal assembly is disposed on the other clamping plate. When the electrode ear clip 22 is energized, the electromagnetic assembly generates a magnetic force, and the two clamping sheets are close to each other under the action of the magnetic force, so as to clamp the ears of the human body. When the electrode ear clip 22 is powered down, the electromagnetic assembly loses magnetic force, and the two clamping sheets loosen the ears of the human body. It should be noted that a torsion spring can also be arranged between the two clamping sheets, so that the two clamping sheets reset the ears of the human body. Therefore, in this embodiment, the electromagnetic assembly is arranged, so that the electrode ear clip 22 can release the ear when the electric pulse is turned off, therefore, the ear of the human body cannot be clamped all the time, discomfort is not caused, and the use experience of the user is improved.

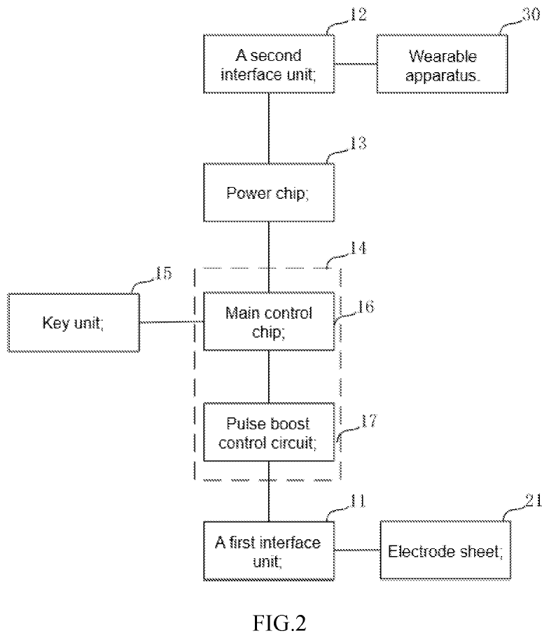

Referring to FIG. 2, in an embodiment, the pulse unit comprises a main control chip 16 and a pulse boost control circuit 17, the main control chip 16 is connected to the power chip 13, and the main control chip 16 is connected to the second interface unit 12 via the pulse boost control circuit 17. Specifically, the main control chip 16 is an MCU, and the MCU is used as a control assembly of the control assembly 10 to control other assemblies to operate. The pulse boost control circuit 17 is used for generating an electric pulse, and may specifically be a power supply provided by an external power source through a boost, a variable-current, alternating-current, and a constant-current repulse output. In other embodiments, the boost may also be a direct pulse output. There are a plurality of specific forms of the pulse boost control circuit 17, as long as an electric pulse can be generated, which is not limited herein.

In an embodiment, the control assembly 10 further comprises a key unit 15, the key unit 15 is connected to the main control chip 16, and the key unit 15 is used for triggering a control signal to cause the main control chip 16 to adjust the electric pulse according to the control signal. Specifically, the key unit 15 is provided with a “+” and a “−” key, which improves the degree of stimulation of the electric pulse by pressing “+” and “−”, or changes the different massage module, or manually controlling the electric pulse by pressing a button combination (such as a long press, a short press, and a continuous pressing).

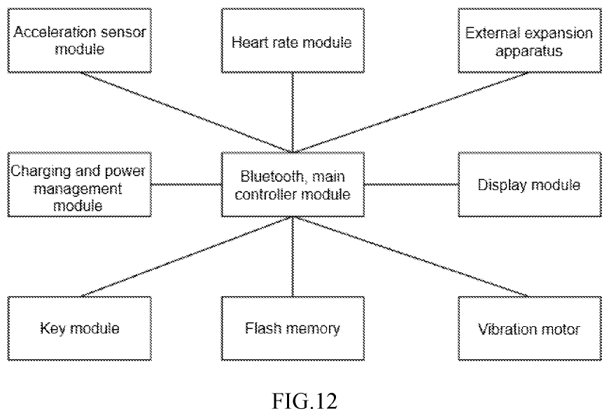

Referring to FIG. 12, in an embodiment, the control assembly 10 comprises a circuit board, the circuit board is provided with various modules of a control assembly, and the circuit board is further provided with bluetooth, a main controller module, a flash memory, a display module, a key module, a charging and power management module, an acceleration sensor module, a vibration motor, a heart rate module, and an external expansion apparatus.

The embodiments of the utility model further provides an electric pulse massage system, including: The wearable apparatuss 30 and the electric pulse massage apparatuss are the electric pulse massage apparatus described in the above embodiments, the wearable apparatuss 30 is connected to the first interface unit 11, and the wearable apparatuss 30 is used for providing a power supply.

Specifically, the wearable apparatus 30 is a smart watch or a smart wristband. In the embodiment, the smart watch or the smart wristband is worn to the wrist of the human body, and the control assembly 10 is connected to the smart watch or the smart wristband by means of the first interface unit 11 (for example, a Type-C interface, a USB interface, a lightning interface, etc.). The control assembly 10 is powered by the smart watch or the smart bracelet. The control assembly 10 can output electrical pulses to the massage assembly 20, and the massage assembly 20 transmits electrical pulses to the human body to perform electrical stimulation massage on the human body. In the embodiment, the smart watch or the smart wristband is used as a power supply apparatus, so that the convenience of the power supply is greatly improved, the convenience of the whole massage apparatus is improved, the convenience of the whole massage apparatus is improved, and the smart watch is not limited by site, is not limited by action, can be used by a human body, sitting and standing, can be used, and is flexible in use mode.

In other embodiments, the wearable apparatus 30 may also be wirelessly connected to the control assembly 10, for example, may be wirelessly connected via bluetooth or RF radio frequency. The control assembly 10 can receive electric energy in a passive manner, and is more convenient and more beautiful to use or store relative to a wired manner, and is more convenient to use and take in.

In other embodiments, a pulse generator may be provided in the wearable apparatus 30 to directly output a pulse to the massage assembly. In this embodiment, the wearable apparatus 30 only provides a power supply for the control assembly.

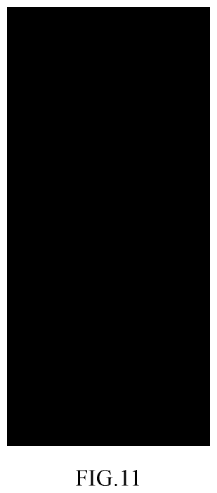

Referring to FIG. 11, in an embodiment, the wearable apparatus 30 comprises a monitoring module, the monitoring module is in communication connection with the pulse generation unit the monitoring module is used for monitoring a physiological parameter electric signal or a human body, and the pulse generation unit 14 is used for adjusting an electric pulse according to the physiological parameter electric signal. The monitoring module includes at least one of a heart rate sensor, a pulse sensor, a blood pressure sensor, a respiration sensor, and a temperature sensor. In the embodiment, the smart watch or the smart wristband is provided with a plurality of sensors, and various physiological parameters of the human body are measured by sensors, such as heart rate, pulse, blood pressure, respiration, temperature, etc. Exemplarily, the heart rate of the human body is usually about 60-100, and when the heart rate is lower than when the heart rate is lower, it indicates that the user may be in a sleep state at this time, when the electric signal detected by the heart rate sensor fluctuates, the waveform of the electrical signal is preset in the MCU as a preset waveform, that is, when the electrical signal detected by the heart rate sensor is in a sleep state, the MCU then stops the electric pulse output, thereby preventing the human body from sleeping or performing electrical stimulation massage.

The MCU is used for adjusting at least one of a current, a voltage, a pulse width, a duty cycle, a frequency and a period of an electric pulse to obtain an adjusted electrical pulse. In the embodiment, for the adjustment of the electric pulse, the adjustment may be performed by using parameters such as current, voltage, pulse width, duty cycle, frequency, period, etc. It should be noted that the number of parameter adjustments is not limited, that is, the current may be adjusted separately, or the current and voltage may be adjusted at the same time, or the current, voltage and pulse width may be adjusted at the same time, which is not limited herein. Exemplarily, the MCU is used for adjusting or decrease the magnitude of the voltage and/or current of the electric pulse according to the amplitude of the physiological parameter electrical signal. The amplitude of the physiological parameter electrical signal is also the magnitude of the physiological parameter electrical signal, for example, the pulse is 70, the heart rate is 80, etc. Exemplarily, when the pulse is monitored to be 100, it is indicated that the human body is in a hyperactive state, so that the degree of electrical stimulation can be improved, and correspondingly, the size of the high current and the voltage can be adjusted.

In summary, on the one hand, the electric pulse massage system of the present embodiment can cooperate with the wearable apparatus 30 to use the wearable apparatus 30 as a power supply, and the control assembly 10 is directly connected to the wearable apparatus 30, so that a wireless connection or wired connection mode can be adopted, a special heavy power supply does not need to be used, and the electric pulse massage system is more convenient and flexible to use. On the other hand, the electric pulse can be automatically adjusted according to the physiological state of the human body by using the monitoring module on the wearable apparatuss 30, and the electric pulse can be adjusted by the parameters that monitoring module monitors. In particular, the monitoring module can automatically stop the electric pulse output according to the physiological state of the human body, avoid the continuous output of the electric pulse to the human body, influence the human health safety, automatically close the electric pulse output by monitoring the state of the human body, improve the safety and reliability of the product, and improve the user experience.

The above description is only the preferred embodiments of the utility model and is not intended to limit the utility model, and any modifications, equivalent substitutions and improvements made within the spirit and principle of the utility model shall within the protection scope of the utility model.

What is claimed is:

1. The electric pulse massage apparatus, comprising:
The control assembly comprises a first interface unit, a second interface unit, a power chip and a pulse generation unit,
The first interface unit is used for accessing an external power supply, the first interface unit is connected to the pulse generation unit through the power chip, and the power chip is used for providing a power supply to the pulse generation unit, the pulse unit is connected to the second interface unit, and the pulse generation unit is used for generating an electric pulse;
The massage assembly is connected to the second interface unit, and the massage assembly is used for receiving the electric pulse and apply the electric pulse to the human body for massage.

2. The electric pulse massage apparatuss according to claim 1, wherein the massage assembly comprises a positive electrode sheet, a negative electrode sheet and a connection wire, the positive electrode sheet and the negative electrode sheet are connected to the second interface unit through the connection wire, the positive electrode sheet and the negative electrode sheet are used for being attached to the surface of a human body, and the electric pulse is output to the human body for massage.

3. The electric pulse massage apparatuss according to claim 2, wherein each of the positive electrode sheet and the negative electrode sheet comprises a centrifugal film layer, a conductive gel layer, a conductive carbon film layer and a fabric layer, and the centrifugal film layer, the conductive gel layer, the conductive carbon film layer and the fabric layer are sequentially stacked;
Wherein, an outer contour of the conductive gel layer matches an outer contour of the fabric layer, an outer contour of the conductive carbon film layer is smaller than an outer contour of the fabric layer, so that a gap is provided between an outer contour of the conductive carbon film layer and an outer contour of the fabric layer, and a width of the gap is 0.8 mm to 1.2 mm.

4. The electric pulse massage apparatuss according to claim 3, wherein the massage assembly further comprises a conductor and a conductive coating film, a through hole is formed in the fabric layer and the conductive carbon film layer, the conductor is disposed on the through hole, and the conductive coating film covers the conductor and is connected to the conductive carbon film layer.

5. The electric pulse massage apparatuss according to claim 1, wherein the massage assembly comprises a positive electrode ear clip and a negative electrode ear clip, and a connection wire, the positive electrode ear clip and the negative electrode ear clip are connected to the second interface unit through the connection wire, the positive electrode ear clip and the negative electrode ear clip are used or clamping the human ear, and the electric pulse is output to the human ear for massage.

6. The electric pulse massage apparatuss according to claim 5, wherein the massage assembly further comprises a conductor, the positive electrode ear clip and the negative electrode ear clip both comprise a first clamping piece and a second clamping piece, and the first clamping piece and the second clamping piece can be close to or far away from each other to clamp or loosen a human ear; The first clamping piece and the second clamping piece are both provided with the conductors.

7. The electric pulse massage apparatus according to claim 1, wherein the pulse unit comprises a main control chip and a pulse boost control circuit, the main control chip is connected to the power chip, and the main control chip is connected to the second interface unit by means of the pulse boost control circuit.

8. The electric pulse massage apparatus according to claim 7, wherein the control assembly further comprises a key unit, the key unit is connected to the main control chip, and the key unit is used for triggering a control signal to cause the main control chip to adjust the electric pulse according to the control signal.

9. The electric pulse massage system, comprising: A wearable apparatuss and an electric pulse massage apparatuss, wherein the electric pulse massage apparatuss is the electric pulse massage apparatuss according to claim 1, the wearable apparatuss is connected to the first interface unit, and the wearable apparatuss is used for providing a power supply.

10. The electric pulse massage system according to claim 9, wherein the wearable apparatuss comprises a monitoring module, the monitoring module is in communication connection with the pulse generation unit, the monitoring module is used for monitoring a physiological parameter electric signal of a human body, and the pulse generation unit is used for adjusting an electric pulse according to the physiological parameter electric signal.
